(19) United States
(12) Patent Application Publication
(10) Pub. No.: US 2008/0104720 A1
(43) Pub. Date: May 1, 2008

(54) Animal Models for Cell Therapy and Cell Based Gene Therapy

(75) Inventors: Reinhold G. Erben, Gaaden (AT); Nina Balke, Biberach (DE); Karin Weber, Prittlbach (DE)

Correspondence Address: FULBRIGHT & JAWORSKI L.L.P., 600 CONGRESS AVE., SUITE 2400, AUSTIN, TX, 78701, US

(73) Assignee: VETERINARMEDIZINISCHE UNIVERSITAT WIEN, Vienna (AT)

(21) Appl. No.: 11/912,856

(22) PCT Filed: Apr. 28, 2006
(86) PCT No.: PCT/AT06/00177

§ 371 Date: Oct. 26, 2007

(30) Foreign Application Priority Data
Apr. 28, 2005 (EP) 05009342.6

(51) Int. Cl. G01N 33/00

(52) U.S. Cl. 800/3

(57) ABSTRACT

The present invention describes the use of marker tolerant animals as a cell-tracking model system for the study of cell based therapies.

The present invention relates to diagnostic models for cell therapy.

Cell and gene therapy are thought to revolutionise tissue repair in various organs, cancer therapy, and therapy of genetic diseases in the near future (1). Therefore, cell and gene therapy holds great promise to cure or ameliorate a large variety of diseases of various organ systems. Cell therapy involves the syngeneic (genetically identical), allogeneic (different individuals of the same species), or xenogeneic (between species) transplantation of cells for therapeutic purposes.

Potential therapeutic targets for cell and gene therapy are:
Liver diseases (for example cell therapy of liver cirrhosis or other severe liver diseases)
Lung diseases (for example cell-based gene therapy of cystic fibrosis)
Skin diseases (for example cell therapy of skin defects with epidermal cells)
Cardiovascular diseases (for example cell therapy of myocardial infarction with mesenchymal cells, gene therapy of cardiomyopathies, for example with vascular growth factors)
Kidney diseases (for example cell therapy or cell-based gene therapy of glomerulonephritis or of renal failure)
Orthopedic diseases (for example cell therapy of osteoarth ritis, disc degeneration, ligament injuries, or cell-based gene therapy of genetic myopathies)
Diabetes mellitus (for example cell therapy of diabetes by transplantation of autologous beta cells grown from precursor cells extracted from pancreas, liver, or bone marrow)
Diseases of the central nervous system (for example cell therapy or cell-based gene therapy of neurodegenerative diseases such as Parkinson's disease or amyotrophic lateral sclerosis, cell therapy of cerebral infarction (palsy), cell therapy of multiple sclerosis)
Cancer (for example cell therapy with ex vivo conditioned autologous immune cells, cell-based gene therapy with autologous cells expressing for example immunostimulatory cytokines such as interleukin 2)

It is clear that any thorough evaluation of the efficacy and applicability of cell and gene therapy methods requires animal models that allow tracing the fate of individual transplanted donor or manipulated cells in the host organism. In order to trace individual cells, it is necessary that these cells are somehow labelled. The labelling is usually done by introducing marker genes into the genome of the cells under investigation. Marker genes are genes whose permanent or transient expression can be used to label cells. Marker genes can either be permanently integrated into the genome of transgenic animals so that all or at least some somatic cells are permanently labelled, or normal cells can be transduced with vectors containing the marker gene. Suitable marker genes are for example Escherichia coli lacZ, green fluorescent protein (GFP) from the jellyfish Aequorea victoria, human placental alkaline phosphatase (hPLAP), or the neomycin phosphotransferase.

During recent years, it has become increasingly clear that membrane or even intracellular expression of any foreign protein, and, thus, of any marker gene, will elicit immune-mediated rejection of the cells carrying the marker gene in the recipients (7-11).

Therefore, immune-mediated rejection of genetically altered cells is a general and very significant problem in transplantation studies using cells labelled with any marker gene, and also in gene therapy protocols. This problem has severely hampered the utility of animal models aimed at testing the usefulness of cell and gene therapy especially in long-term studies. There are several solutions to this problem of immune-mediated rejection of transplanted, labelled cells:

The host can be treated with immunosuppressive drugs such as cyclosporins or glucocorticoids (12). It is clear that with this approach it is difficult to tell whether immune-mediated phenomena were actually absent or not. In addition, immunosuppressive drugs have to be introduced into the experimental system, possibly influencing the outcome of the experiments.

Cells of another species (e.g. human cells) can be transplanted into mice or other animals suffering from severe immune defects such as severe combined immunodeficiency (SCID). SCID mice are unable to reject foreign tissues. Problems with this experimental system are that SCID mice are difficult to keep and that they are a highly artificial system due to the severe immune defects. Although animals have been made available in the art which have been made tolerant against specific selection markers or expression vector encoded components, such animals have always been used for inducing tolerance to transplant tissue or cells. However, it was not suggested to provide a general model system for such processes.

Accordingly, such a model system for observing and studying the performance and effectiveness of a cell based therapy based on genetic molecular markers which would not be biased by unnatural effects, such as immunologic side effects would offer significant advantages for the present field, especially as a research tool.

Therefore, the present invention provides the use of marker tolerant animals as a diagnostic model for the tracking of cells within the context of cell therapy. The present invention provides a model system for cell tracking which allows the study and tracking of cells which contain a suitable marker without any bias with respect to such markers, i.e. without that the marker would have a negative effect or otherwise influence the reaction of the model animals—except of course the cells to be studied.

Since the present invention provides for an animal model, it is clear that the term “animal” exclusively refers to non-human animals as far as the model animal is concerned.

The present solution to the aforementioned problem consists of an entirely different approach as the prior art strategies: In the model which is established by the present invention animals are first made immunologically tolerant to the marker gene that allows tracing of cells to be transplanted into the animals wherein the presence and movement of the cells will be analysed. In a second step, cell and gene therapies with cells labelled with the same marker gene can be tested in the complete absence of immune-mediated rejection. Thus, this model is almost fully equivalent to the situation in human patients using autologous adult stem cells for cell therapy. Prior art exclusively deals with providing tolerance induction in animals for testing whether a gene therapy may be successfully applied (French et al., Diabetes 46(1) (1997), 34-39; Xu et al., Clin. Immunol. 111(1) (2004), 47-52; Ogawa et al., Gene Therapy 1(3) (2004), 292-301; EP 1 142 473 A1). However, studying the route and movement of cells (with a marker) in marker-tolerant animals has neither been suggested nor made obvious by the prior art. Not even the need for having such a cell-tracking model system was present in the art.

In humans, clearly the most practical and applicable method for cell and gene therapy is therapy with autologous stem cells. The general steps of such a therapy can be outlined as follows: Stem cells are extracted from the patient (for example from bone marrow), cultivated and expanded, possibly differentiated in a certain direction or manipulated to express certain genes for gene therapy, and these cells are then re-injected into the same patient either systemically or locally for tissue repair or gene therapy. In addition, tissues or organs can be grown from these cells in culture, and the tissues or organs can be re-transplanted into the patient. Therefore, autologous stem cell therapy is not complicated by immune-mediated rejection phenomena as long as the cells are not transduced with genes encoding non-human proteins. Thus, it can be envisaged that any results obtained with the model according to the present invention are highly predictive of the therapeutic potential of autologous cell and gene therapy in humans.

Recently, it has been shown that low amounts of foreign proteins (for example proteins from fetal bovine serum) attached to the cell surface or internalised during culture can elicit an immune response in the host (13). The present invention (especially the protocols E-G described in the example section) is also capable of coping with this problem. When cells for induction of tolerance and cells for later cell therapy are cultivated in an identical fashion, tolerance will also be induced to any antigens stemming from the culture medium.

With the present invention, tolerance against the tracking protein (which is the marker), is induced in an animal which animal is then used as a model for tracking transplanted cells which are detectable (trackable) by the marker (to which the animal is tolerant and to which the animal therefore also does not show any negative reactions, e.g., (immunological) rejection reactions). According to the present invention, tolerance is induced in the animals for the generation of a model for tracking cells (containing the marker to which the animal is tolerant).

The present invention is specifically useful for the evaluation of syngeneic and allogeneic cell therapies. The present invention is most appropriate to study syngeneic and allogeneic cell therapies with mesenchymal stem cells. Cell therapy also includes cell-based gene therapy involving the transplantation of cells transduced to express certain genes for therapeutic purposes.

Further aspects of the present invention are
a kit for investigating cell therapy comprising a marker tolerant animal and a cell comprising the marker and
a method for studying the applicability of cells in cell-therapy comprising the steps of providing said cells to be studied with a marker, transplanting said cells with the marker to an animal, said animal comprising a tolerance against said marker, and studying the applicability of said cells with said marker in cell therapy of said marker tolerant animals, preferably tracking said cells in said animals upon transplantation; wherein said method preferably (i.e. usually) comprising killing said animals.

The following preferred embodiments relate to the use, the kit as well as the method according to the present invention, wherever appropriate.

Preferably, the marker tolerant animals according to the present invention are lethally irradiated inbred or cloned animals transplanted with bone marrow cells expressing said marker, animals having been exposed neonatally to said marker or animals having been exposed postnatally to marker cells by intravenous infusion.

Specifically preferred are animals which are provided by the following method steps:
providing said cells to be studied with a marker,
transplanting said cells with the marker to an animal, said animal comprising a tolerance against said marker, and
studying the applicability of said cells with said marker in cell therapy of said marker tolerant animals, preferably comprising killing said animals.

The marker to be used is preferably a marker which is already established in cell therapy and transgenic animals. The markers according to the present invention are, of course, markers which are—in general—immunogenic for the individual animal as such and for which therefore tolerance has to be established in order to prevent the negative consequences of immune reactions for the tracking process. It should be easily detectable upon expression and not be harmful in the animal to be studied. Most suitable markers can be selected from Escherichia coli lacZ, green fluorescent protein (GFP) or human placental alkaline phosphatase (hPLAP). Preferred markers according to the present invention are colorigenic markers (e.g. enzymes which catalyse a colour-creating reaction), fluorigenic markers, biophotonic markers (e.g., light-producing enzymes, luciferases, reviewed in Doyle et al., Cellular Microbiology 6: 303-317, 2004, or Contag et al., Annu Rev Biomed Eng 4: 235-260, 2002), positron emission tomography markers (e.g., marker enzymes such as herpex simplex virus 1 thymidine kinase which metabolize and, thus, trap, molecules labeled with positron-emitting radioisotopes; e.g., Gambir S S et al., PNAS 96: 2333-2338, 1999), nuclear magnetic resonance imaging markers (e.g. protein markers increasing the metal ion content of cells such as ferritin, Genove G et al., Nature Medicine 11, 450-454, 2005), or other marker which allow immediate detection, especially optical detection (by colours, fluorescence, etc.) On the other hand, the markers should not interfere with the cell based therapy which is observed in the model. Therefore, markers which are or are related to transplantation antigens, autoimmune antigens, components of the immune system, therapeutically active proteins or peptides or metabolically active proteins are not preferred according to the present invention and completely unsuitable if they confer negative influence on the trackability of the cells in the individual model system (i.e. if the marker interferes with the action of the cells in a disturbing or otherwise negative manner).

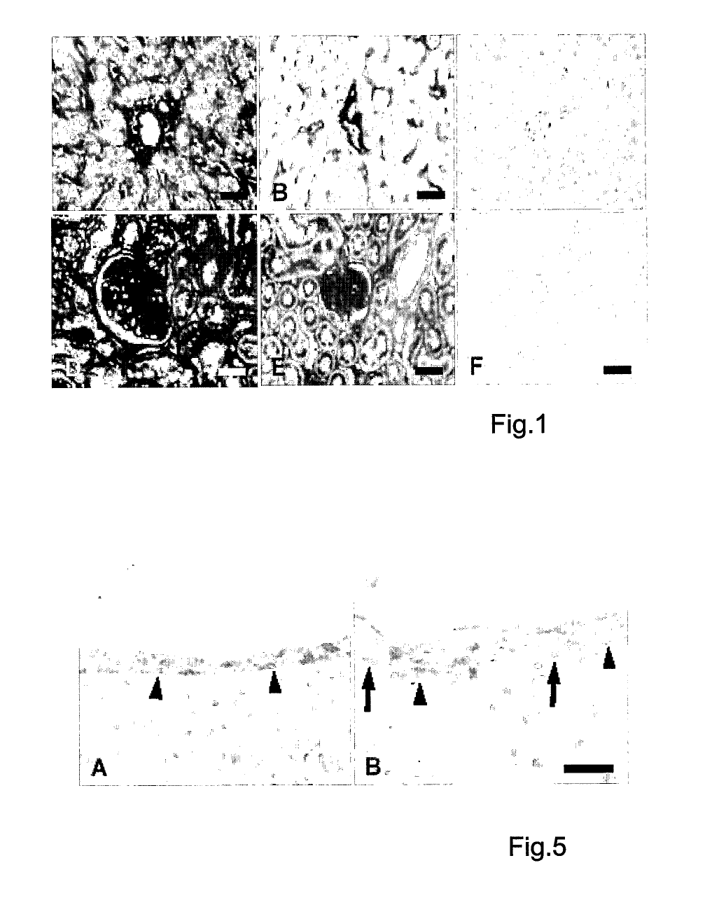

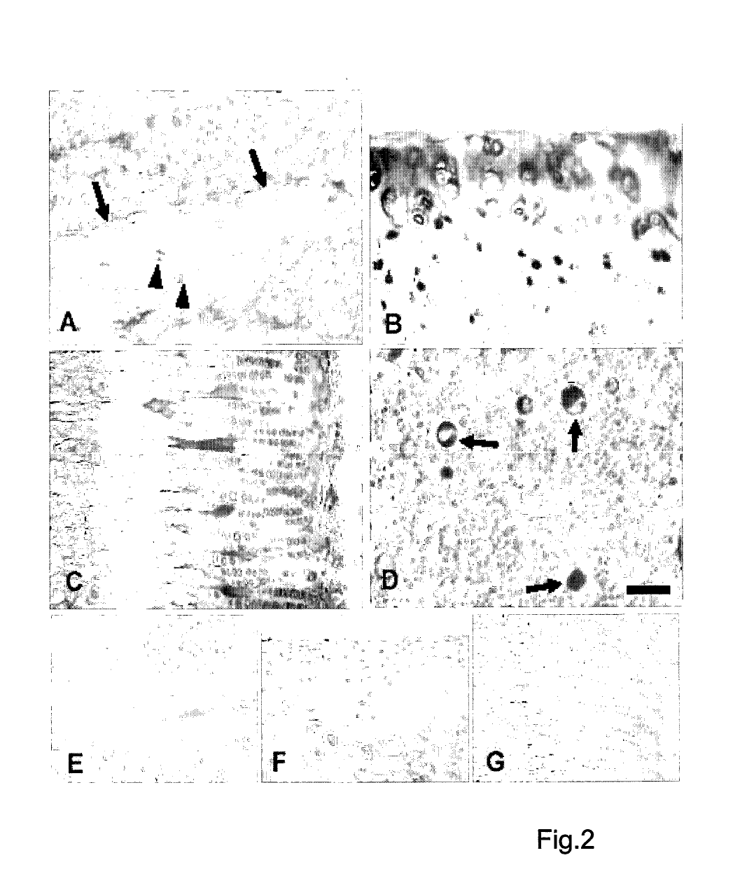

The preferred marker genes for studies involving cell and gene therapy—lacZ, GFP and hPLAP—are known for their suitable detectability in histological sections. LacZ can easily be detected histochemically, but is heat labile, and the enzyme activity is destroyed during paraffin embedding (2). Therefore, pre-embedding staining methods or immunohistochemistry are necessary to visualise the marker lacZ. GFP can easily be visualised in living cells because of its fluorescent properties (3). However, the fluorescent properties of GFP are greatly diminished during normal paraffin embedding, and sensitive detection of GFP in paraffin sections requires either sophisticated equipment (4) or immunohistochemical detection methods (5). Thus, the usefulness of GFP for histological studies is limited. hPLAP is a marker gene highly suitable for histological studies requiring tissue sectioning and excellent tissue morphology, because hPLAP is a heat-stable enzyme that retains its enzymatic activity during routine paraffin embedding (6). In addition, it could be shown with the present invention that hPLAP enzyme activity is pre-served during methylmethacrylate (MMA) embedding (FIG. 1). Enzymatic activity of hPLAP can easily and very sensitively be detected histochemically or immunohistochemically in tissue sections by post-embedding procedures (FIG. 1). Therefore, hPLAP is one of the most suitable marker genes to date for the evaluation of cell and gene therapies. On the other hand, it was also found in the course of the present invention that the genetic marker hPLAP induced strong humoral immune responses in wild-type recipient rats, resulting in the rejection of intravenously infused cells from transgenic donors (FIG. 2), also proving that the need for an absolutely marker tolerant animal is important for providing a non-biased model.

The preferred fields of use of the present animal model are the tracking of cells in the context of cell therapies for stroke, multiple sclerosis, neurodegenerative disorders, arthroses, intervertebral disc degeneration, ligament injuries, myopathies, myocardial infarction, cardiomyopathies, glomerulonephritis, severe liver cell damages, epidermal cell therapy or cancer, including the generation and transplantation of tissues or organs grown from individual cells in culture.

Preferably, the animal to be used according to the present invention is a rodent, especially a rabbit, a hamster, a mouse, a guinea pig or a rat, a primate, especially a chimpanzee, or a pig.

Depending on the model or on the cells to be studied, the marker in the cell is controlled by a specific promoter. A preferred promoter is an inducible promoter (reviewed by Romano G Systems for regulated or tissue-specific gene expression. Drug News Perspect 17:85-90; Clackson T Regulated gene expression systems. Gene Therapy 2000, 7:120-125; Pollock R and Clackson T Dimerizer-induced gene expression. Current Opin Biotechnol 2002; 13:459-467). In other preferred embodiments, the promoter is a ubiquitous constitutive promoter, especially ROSA26 (R26) promoter, human, rat, or chicken β-actin promoter or β-actin promoter with cytomegalovirus enhancer, cytomegalovirus promoter, ubiquitin promoter, or SV40 promoter. In other preferred embodiments, the promoter is a cell- or tissue-specific constitutive promoter targeting marker expression to specific hematopoietic cells such as the megakaryocytic/platelet-specific alphaIIb promoter (16), the lymphoid cell-specific hCD2 promoter (17) or the macrophage-specific c-fms gene promoter (18). A specific advantage of a constitutive promoter is that all cells derived from a genetically labeled cell express the marker regardless of differentiation status. A specific advantage of an inducible promoter and a cell- or tissue-specific promoter in the current invention is that marker gene expression can be limited in space and/or time. Using cells expressing the marker under control of a tissue-specific or inducible promoter for induction of tolerance, a marker tolerant animal can be generated without background expression of labeled cells in the marker tolerant animal, or with background expression only in cells not interfering with the analysis of applicability. A further specific advantage of an inducible promoter in transplanted cells is that cell-based gene therapy can be monitored if the marker and the therapeutic gene are under the control of the same inducible promoter.

The preferred cells to be applied in the present cell-tracking model for cell therapy according to the present invention is a therapy employing mesenchymal cells, especially osteoblasts, chondrocytes, adipocytes, fibroblasts, myoblasts or combinations thereof.

Other preferred forms of cell therapy to be studied with the present model are a therapy employing non-hematopoietic cells selected from epithelial cells, endothelial cells, liver cells, pancreas cells, neuronal cells or combinations thereof.

According to a preferred embodiment of the present invention, the cells with the marker are transplanted into deficient parts of the mammal selected from the group consisting of heart, central nervous system (CNS), bone, cartilage, joint, lung, intestine, liver or pancreas.

Preferably, the marker tolerant animal in the model or kit of the present invention is a rodent, especially a rabbit, a hamster, a mouse, a guinea pig or a rat, a primate, especially a chimpanzee, or a pig.

Preferred cells comprising the marker are differentiated or partially differentiated mesenchymal cells, especially mesenchymal cells contained in bone marrow or mesenchymal cells derived from bone marrow stem cells. However, also mesenchymal cells from other tissue (skin, liver, . . . ) are preferred.

According to a preferred embodiment, the kit according to the present invention further comprises 1) a marker tolerant animal, 2) cells comprising the marker, 3) a transfection vector to transduce cells with the marker, 4) a detection system for the marker, for example in histological sections.

In the method for studying the applicability or movement of cells in cell-therapy according to the present invention, the following steps are applied:
providing said cells to be studied with a marker,
transplanting said cells with the marker to an animal, said animal comprising a tolerance against said marker, and
studying the applicability or movement of said cells with said marker in cell therapy of said marker tolerant animals, preferably tracking said cells in said animals upon transplantation; wherein said method preferably comprises killing said animals.

As mentioned above, the marker tolerant animals may be provided by any suitable technique, the most preferred being selected from the group of inbred animals, cloned animals, bone marrow marker cells transplanted animals, animals having been exposed neonatally to said marker or animals having been exposed postnatally to marker cells by intravenous infusion.

The present invention also provides a specific technique for providing excellently suitable marker tolerant animals, wherein the marker tolerant animals are provided by a method which comprises the steps of
obtaining a transgenic mammal expressing a marker protein under the control of a promoter,
isolating bone marrow cells (BMCs) from the transgenic mammal,
irradiating a wild type mammal of the same species and
injecting the isolated BMCs into the irradiated wild type mammal,
   before transplanting said cells with the marker to the wild type mammal.

The animals obtainable by this specific technique also represent an aspect of the present invention. The specific advantage of lethal irradiation followed by bone marrow transplantation in inbred or cloned animals is that this method represents the most robust approach for induction of lifelong and complete immunological tolerance to the marker gene.

The present invention is further described by the following examples and the drawing figures, yet without being restricted thereto.

FIG. 1 shows paraffin and methylmethacrylate (MMA) sections of liver and kidney from R26-hPLAP-tg and wild-type rats stained for hPLAP activity after heat pretreatment. In paraffin sections of liver from transgenic rats, hPLAP staining is more intense in specimens fixed with 40% ethanol (A) compared with PFA fixation (B). No staining is observed in ethanol-fixed liver of a wild-type rat (C). The kidney from a transgenic rat shows comparable hPLAP staining intensity in paraffin-(D) and MMA-embedded specimens (E). However, morphological detail is improved in MMA sections, and renal tubular cells show a clear membranous hPLAP staining pattern. Note the ubiquitous hPLAP staining pattern in transgenic tissues. No staining is seen in ethanol-fixed, MMA-embedded kidney of a wild-type rat (F). Sections A—F were stained for hPLAP enzyme activity overnight at room temperature and were counterstained with nuclear fast red. Five-μm-thick sections. Bar=50 μm.

FIG. 2 shows ubiquitous expression of hPLAP in bone, cartilage, and bone marrow cells of R26-hPLAP-tg rats (A-D). Osteoblasts (arrows) and osteocytes (arrowheads) show intense membranous hPLAP staining in transgenic rats (A). Chondrocytes in articular and growth plate cartilage are intensely stained (B, C). Hematopoietic bone marrow cells show ubiquitous hPLAP staining with very strong staining on the cell membrane and in the cytoplasm of megakaryocytes (D). No staining is seen in bone or bone marrow (E), articular cartilage (F), or growth plate cartilage from wild-type rats (G). Five-μm-thick undecalcified MMA sections of ethanol-fixed tibias stained for hPLAP activity and counterstained with nuclear fast red. Bar=50 μm.

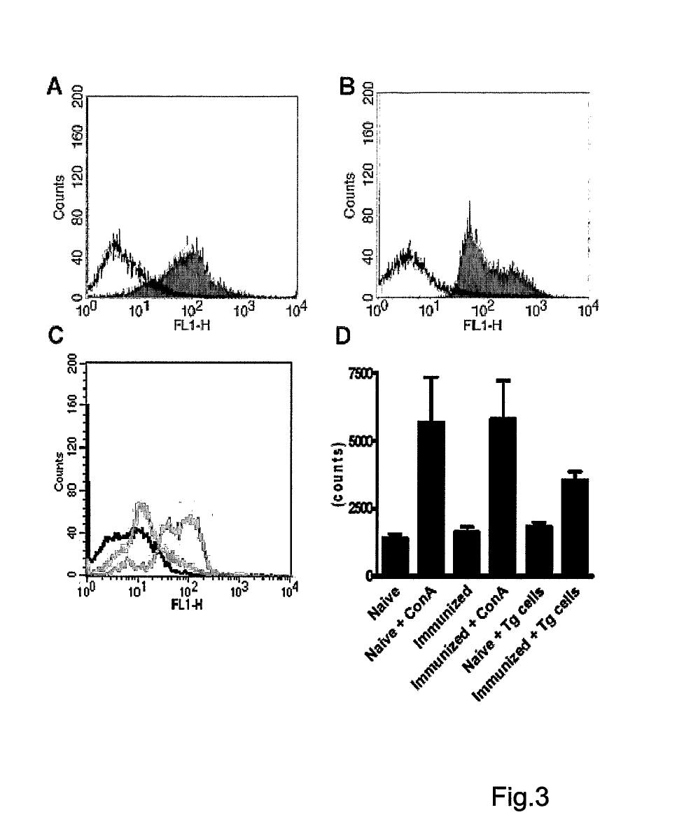

FIG. 3 shows FACS analysis of peripheral blood and bone marrow cells from R26-hPLAP-tg rats and immune response of wild-type rats to transgenic cells. The single parameter fluorescence histograms show hPLAP expression (mAb clone 8B6) on the majority of peripheral blood leukocytes (A) and practically all bone marrow cells (B) in R26-hPLAP-tg rats (grey area), but no expression in wild-type rats (white area). When peripheral blood leukocytes from R26-hPLAP-tg rats are incubated with serum from a naïve wild-type rat, and stained for bound antibodies, no staining is seen (black curve, C). However, serum from the same wild-type intraperitoneally injected with cells from transgenic donors 3 times shows strong reactivity with peripheral blood leukocytes from R26-hPLAP-tg rats (C, green, after 2 injections; red, after 3 injections), indicating the induction of circulating antibodies against transgenic cells in the recipient. Ten-thousand cells were analyzed in each sample. In a mixed lymphocyte reaction protocol (D), co-culture of F344-Tg (R26-hPLAP) spleen cells with spleen cells from wild-type rats previously injected with cells from F344-Tg (R26-hPLAP) rats, but not from naïve wild-type rats, caused increased cell proliferation. ConA was used as a positive control. 5×10⁵ spleen cells per well were cultivated for 5 days (n=8 wells each).

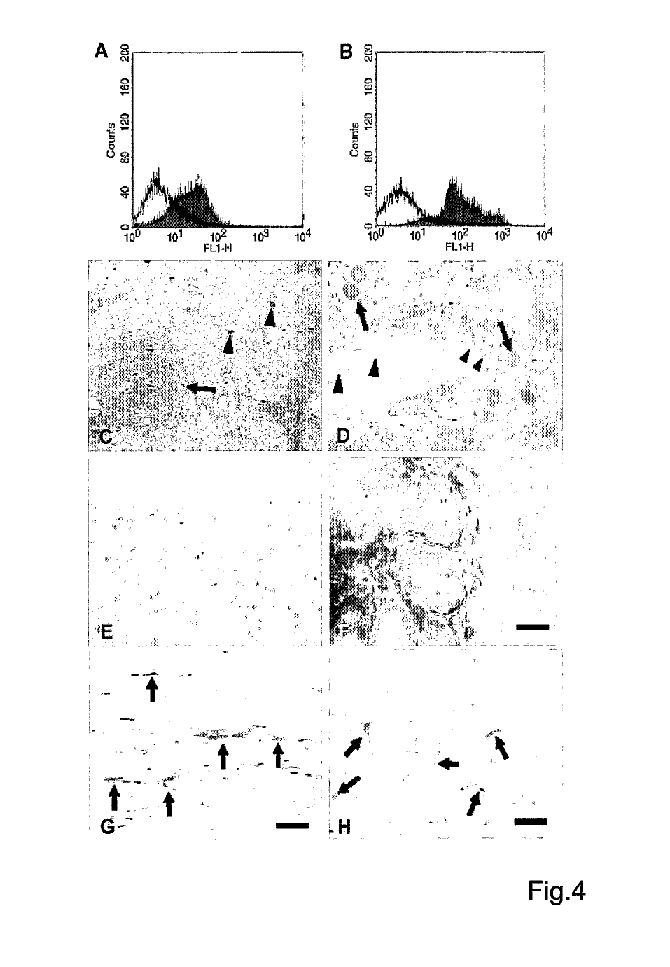

FIG. 4 shows expression of hPLAP in tissues of wild-type rats transplanted with bone marrow from R26-hPLAP-tg rats, 4 weeks post-transplantation. FACS analysis reveals hPLAP expression (mAb clone 8B6) on peripheral blood leukocytes (A) and bone marrow cells (B) in R26-hPLAP bone marrow-transplanted (BMT) wild-type rats (grey area) in contrast to wild-type rats (white area). The staining pattern is very similar to the one observed in R26-hPLAP-tg rats (FIG. 3A-B). Lymphocytes in lymph follicles (arrows) and megakaryocytes (arrowheads) in the spleen demonstrate clear hPLAP staining. Bone marrow cells, especially megakaryocytes (arrows), show ubiquitous hPLAP expression, whereas osteoblasts, osteocytes (D, small and large arrowheads, respectively) and chondrocytes in articular cartilage (E) do not express hPLAP, indicating their host origin. In cross-sections of the ileum, enterocytes and smooth muscle cells remain unstained, while cells in the submucosa and endothelial cells show intense hPLAP labeling (F). Cardiomyocytes (G) and renal tubular epithelial cells (H) are hPLAP negative cells of recipient origin, while a large extent of capillary endothelium in heart and kidney is of donor origin as indicated by hPLAP labeling (arrows). Five-μm-thick paraffin sections of spleen (C), ileum (F), heart (G), and kidney (H), and undecalcified MMA sections of ethanol-fixed tibias (D-E) stained for hPLAP activity and counterstained with nuclear fast red. Bar=50 μm.

FIG. 5 shows evidence for hPLAP-expressing cells on the articular cartilage after injection of R26-hPLAP-tg bone marrow cells into the knee joint of R26-hPLAP BMT wild-type rats. One week post-injection, there is a 2-3 cells wide layer of donor cells (arrowheads) on the cartilage surface (A). Four weeks post-injection, transgenic donor cells have adopted a fibroblast-like (arrowheads) or chondrocyte-like (arrows) appearance, and show incorporation into the surrounding chondrocyte matrix of host origin (B). Five-μm-thick undecalcified MMA sections of ethanol-fixed knee joints stained for hPLAP activity and counterstained with nuclear fast red. Bar=50 μm.

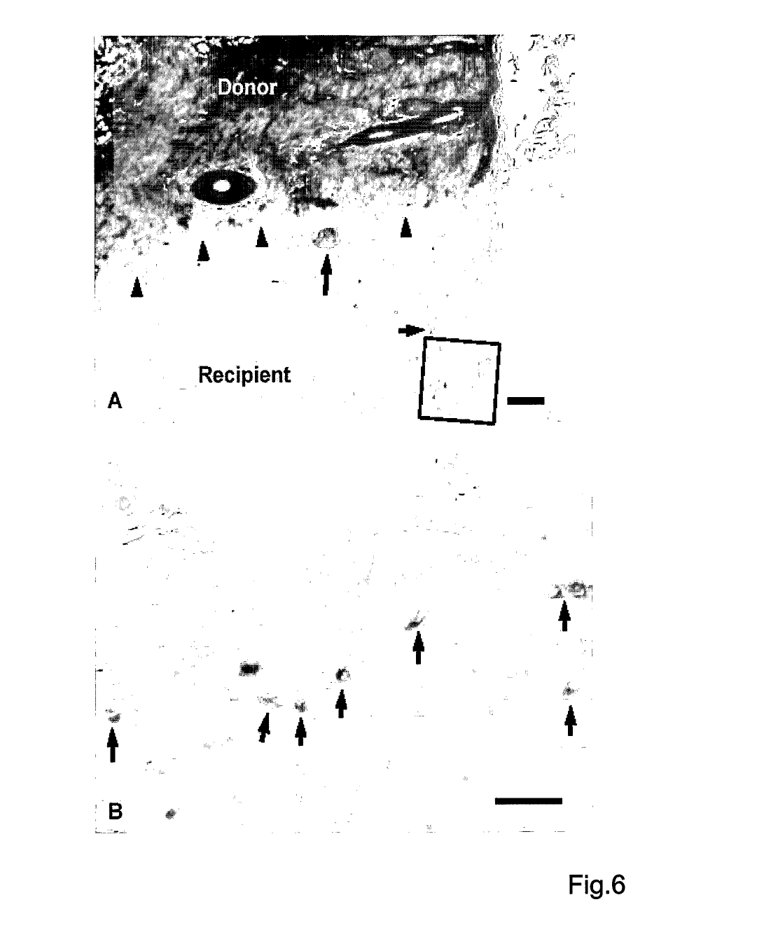

FIG. 6 shows skin from F344-Tg (R26-hPLAP) donor grafted into a neonatally tolerized, 4-month-old F344 wild-type rat, 4 weeks post-transplantation. The skin graft from the transgenic donor (upper part of the section) shows strong expression of hPLAP in all cells (A). The border between transgenic donor tissue and wild-type recipient tissue is sharply delineated (arrowheads). A sebaceous gland (large arrow), and a few capillaries (small arrow in A, arrows in B) of transgenic donor origin are found in the surrounding wild-type skin. Keratinocytes do not cross the sharp border between wild-type and transgenic epidermis. FIG. 6B is a high power view of the frame shown in (A). Five-μm-thick paraffin section of ethanol-fixed skin biopsy stained for hPLAP activity and counterstained with nuclear fast red. Bar=100 μm.

### EXAMPLES

A central aspect of this invention is the induction of immunological tolerance to the marker gene. There is a number of known strategies to induce tolerance to foreign antigens. The most robust approach is to lethally irradiate the host organism, and to transplant bone marrow cells expressing non-self antigens (for example the marker gene) into the lethally irradiated host.

Therefore, this approach was used as proof-of-principle in the current invention. Because all immune cells are of donor origin after irradiation and bone marrow transplantation, the foreign donor antigens are not recognised as non-self by immune cells. However, if donor and recipient are not genetically identical (with the exception of the marker gene) this approach requires lifelong immunosuppression because of the danger of a graftversus-host reaction. The latter situation is, for example, found in leukemia patients after lethal irradiation or chemotherapy and heterologous (between two related, but not genetically identical individuals) bone marrow transplantation. Therefore, we use this approach in the current invention only in genetically identical, inbred or cloned animals. Alternatively, animals can be sublethally irradiated or treated with chemotherapy before bone marrow transplantation, resulting in the generation of chimera. Another strategy is to expose neonatal animals to the foreign antigens. For example, injection of neonates with semi-allogeneic donor cells results in specific tolerance and lifelong survival of skin grafts from the same donor (14). A third strategy is the intravenous infusion of very high numbers of semi-allogeneic donor cells into postnatal hosts (15). In the latter situation, the massive exposure to non-self antigens obviously overrides the immune system so that tolerance is induced. Other methods to induce tolerance to certain antigens are conceivable and could also be used for this invention, for example intranasal application of foreign proteins.

Specifically, the present invention is preferably demonstrated by the application of the following scenarios A to G:

A) Genetic Marker in Inbred Animals (Mice, Rats, Pigs, Rabbits, Hamster)

1. Generation of transgenic animals on an inbred genetic background (for example R26-hPLAP-tg Fischer 344 rats in the present experiments expressing the marker gene under a ubiquitous promoter such as beta-actin, cytomegaly virus, or R26, or a promoter targeting transgene (=marker) expression to specific hematopoietic cells such as the megakaryocytic/platelet-specific alphaIIb promoter (16), the lymphoid cell-specific hCD2 promoter (17) or the macrophage-specific c-fms gene promoter (18).

2. Generation of marker tolerant wild-type animals of the same inbred strain by a) lethally or sublethally irradiating wild-type animals or treating wild-animals with chemotherapy and b) transplanting the irradiated animals with bone marrow cells from transgenic donors. All hematopoietic but not the mesenchymal and epithelial cells in the recipient are now of transgenic donor origin.

3. Transplanting genetically labelled cells from transgenic donors described in 1) into the marker tolerant animals.

4. Studying the applicability of such cells in cell and gene therapy.

B) Genetic Marker in Cloned Animals

This protocol is identical to A) with the difference that genetically identical, cloned animals are used instead of inbred animals.

C) Genetic Marker in Inbred Animals (Mice, Rats, Pigs, Rabbits, Hamster) Using an Inducible Promoter

1. Generation of transgenic animals on an inbred genetic background (for example hPLAP-tg Fischer 344 rats in the present experiments) expressing the marker gene under a ubiquitous promoter such as beta-actin, cytomegaly virus, or R26, or a promoter targeting transgene (=marker) expression to specific cell lineages.

2. Generation of transgenic animals of the same inbred strain expressing the marker transgene under an inducible promoter such as ligand-inducible gene regulatory systems (19). Induction of the transgene, preferably in neonatal animals, results in lifelong tolerance to the marker gene.

3. Transplanting genetically labelled cells from transgenic donors described in 1) into the marker tolerant animals.

4. Studying the applicability of such cells in cell and gene therapy.

D) Genetic Marker in Genetically Non-Identical Animals

1. Generation of transgenic animals on an outbred or inbred genetic background expressing the marker gene under a ubiquitous promoter such as beta-actin, cytomegaly virus, or R26, or a promoter targeting transgene (=marker) expression to specific cell lineages.

2. Generation of wild-type animals of the same species but not of the same inbred strain compared with animals described in 1) that are made tolerant to the marker and the antigens of the donor cells by neonatal or postnatal exposition to transgenic cells. Preferably, the animals described in 1) and 2) should be closely related, and should have some common MHC class antigens.

3. Transplanting genetically labelled cells from transgenic donors described in 1) into the marker tolerant animals described in 2).

4. Studying the applicability of such cells in cell and gene therapy.

E) Use of Autologous Cells Transduced with the Marker Gene for Cell Therapy in Marker Tolerant Animals of any Species

1. Generation of marker tolerant animals by a) taking cells from a neonatal or postnatal animal, b) transducing the cells with the marker gene, and c) re-injecting the transduced cells into the neonatal (preferred) or postnatal (very high cell numbers) host to induce tolerance against the marker gene.

2. Transplanting autologous cells from the same animal described in 1) by a) extracting cells from the host, b) transducing these cells with the same marker gene described in 1), and c) transplanting these autologous, transduced cells into the tolerant host.

3. Studying the applicability of such cells in cell and gene therapy.

F) Use of Cells Transduced with the Marker Gene for Cell Therapy in Marker Tolerant Animals of the Same Inbred Strain

1. Generation of inbred, marker tolerant animals by a) taking cells from an animal of the same inbred strain, b) transducing the cells with the marker gene, and c) re-injecting the transduced cells into the neonatal (preferred) or postnatal hosts.

Alternatively, marker tolerant inbred animals can be obtained by the methods described in A) or C).

2. Transplanting cells from wild-type animals of the same inbred strain described in 1) by a) extracting cells from these animals, b) transducing these cells with the same marker gene described in 1), and c) transplanting these transduced cells into the tolerant hosts of the same inbred strain.

3. Studying the applicability of such cells in cell and gene therapy.

This procedure equals E) with the difference that not autologous cells, but cells of the same inbred strain, are used.

G) Use of Cells Transduced with the Marker Gene for Cell Therapy in Marker Tolerant Cloned Animals

1. Generation of cloned, marker tolerant animals by a) taking cells from an animal of the same clone, b) transducing the cells with the marker gene, and c) re-injecting the transduced cells into neonatal (preferred) or postnatal hosts of the same clone. Alternatively, marker tolerant cloned animals can be obtained by the methods described in B).

2. Transplanting cells from animals of the same clone described in 1) by a) extracting cells from these animals, b) transducing these cells with the same marker gene described in 1), and c) transplanting these transduced cells into the tolerant hosts of the same clone.

3. Studying the applicability of such cells in cell and gene therapy.

This procedure is identical to F) with the difference that not animals from an inbred strain, but cloned animals are used.

It is clear that the studying step is usually connected with killing said model animal or at least disrupting the physiology when assessing the therapy so that a therapeutic effect to the test animal is not reached in the end.

### Example 1

### Injection of Bone Marrow Cells from R26-hPLAP-Trans-Genic Inbred Fischer 344 Rats into the Knee Joint of Marker Tolerant Inbred Fischer 344 Wild-Type Rats

Bone marrow cells from R26-hPLAP-transgenic inbred Fischer 344 rats were injected into the knee joint of marker tolerant inbred Fischer 344 wild-type rats. Tolerance to hPLAP was induced in the wild-type rats by lethal irradiation and bone marrow trans-plantation with R26-hPLAP-transgenic bone marrow (methodology described in A). One week post-injection, the transgenic BMC had formed a dense, 2-3 cells wide, layer on the intact articular cartilage surface. This finding shows that these cells underwent proliferation on the cartilage surface earlier. After 4 weeks, the genetically labelled cells displayed a more differentiated fibroblast- or chondrocyte-like phenotype and appeared to have become integrated into the surrounding cartilage tissue of wild-type origin. This experiment underscores the potential usefulness and strengths of this model, and clearly demonstrates that mesenchymal precursors obviously present in native bone marrow can adhere to the articular cartilage surface and can, subsequently, differentiate into a fibroblastic or chondrocytic phenotype. Thus, this invention can be used to validate and optimise cell therapies aimed to cure or ameliorate, for example, osteoarthritis.

### Example 2

### Myocardial Infarction Model

After generation of hPLAP tolerant animals according to protocols A-G, a coronary artery is ligated as a myocardial infarction model. At various time points after the ligation, hPLAP-labelled, cultured mesenchymal stromal cells or more differentiated cells prepared by protocols A-G are intravenously infused. At various time points after the infusion, the animals are killed, and the presence of hPLAP-labelled cells is analysed by histochemistry or immunohistochemistry in the heart. Similar experiments have been described in normal rats with marrow stromal cells expanded in culture and labelled with a reporter gene (20). However, as mentioned above, these experiments are marred by uncertainties regarding immune-mediated rejection phenomena.

### Example 3

### Traumatic Brain Injury Model

After generation of hPLAP tolerant animals according to protocols A-G, a traumatic brain injury is induced. At various time points after the injury, hPLAP-labelled, cultured mesenchymal stromal cells or more differentiated cells prepared by protocols A-G are intravenously infused. At various time points after the infusion, the animals are killed, and the presence of hPLAP-labelled cells is analysed by histochemistry or immunohistochemistry in the brain. Similar experiments have been described in normal rats using human bone marrow stromal cells (21). However, as mentioned above, these experiments are marred by uncertainties regarding immune-mediated rejection phenomena.

### Example 4

### Marker Tolerant Animals as Models for Regenerative Therapies and Long-Term Cell Tracking

Methods

Animals. All experimental procedures were conducted in compliance with prevailing animal welfare regulations. Heterozygous male or female R26-F344 hPLAP-tg rats were mated with wild-type F344 rats, and the resulting wild-type and heterozygous transgenic offspring were genotyped by enzyme histochemistry using a drop of tail blood as described (6). The rats were housed in pairs at 24° C. and a 12 h/12 h light/dark cycle with free access to tap water and commercial rat diets (Altromin 1324 for maintenance and 1314 for breeding, Altromin, Lage, Germany).

Histology and hPLAP histochemistry. To establish the most useful method of fixation and tissue processing, liver, kidney, and bones from adult wild-type and R26-hPLAP-tg animals were harvested and were fixed in 4% paraformaldehyde (PFA) in 0.1M phosphate buffer at pH 7.4 at 4° C. for 24 h, or in 40% ethanol at 4° C. for 48 h. Subsequently, the tissue specimens fixed in PFA were washed overnight in 0.1M phosphate buffer, pH 7.4, containing 10% sucrose. Fixed tissues were dehydrated and embedded in paraffin or in a modified MMA embedding mixture that preserves enzyme activities, and that can be used for immunohistochemistry (22). Five-μm-thick paraffin and MMA sections were cut with a HM360 microtome (Microm, Walldorf, Germany), and were mounted on slides pre-treated with 3-aminopropyltriethoxy-silane (APES, Sigma-Aldrich, Deisenhofen, Germany).

Paraffin sections were deparaffinated using xylene, whereas MMA sections were deplasticized using 2-methoxyethylacetate as described (22). Deparaffinated and deplasticized sections were rehydrated and heated at 65° C. for 30 min in deionized water to block endogenous alkaline phosphatase activity. Sections were then incubated in TRIS buffer (0.1M Tris-HCl, pH 9.5, 0.1M NaCl, 5 mM MgCl₂) containing 0.17 mg/ml of the substrate 5-bromo-4-chloro-3-indolyl phosphate (BCIP, Sigma) at 37° C. for 1, 2, 3, 4, 5 or 6 h, or at room temperature overnight. Subsequently, sections were counterstained with nuclear fast red (Sigma), dehydrated, and mounted using Vectamount (Vector, Burlingame, Calif., USA).

Immunohistochemical detection of hPLAP expression in transgenic rats was performed on PFA- or ethanol-fixed paraffin- and MMA-embedded tissues. Sections from wild-type animals were used as controls. Sections were deparaffinized or deplasticized, and, after blocking with 20% horse serum, incubated for 2 hours at room temperature with a monoclonal mouse IgG₂ₐ anti-hPLAP antibody (supernatant of clone 8B6, Dako, Hamburg, Germany) diluted 1:50. Bound antibody was detected with biotinylated horse anti-mouse IgG and peroxidase-conjugated avidin biotin complex (Vector), using Vector VIP as enzyme substrate. Sections were dehydrated without counterstaining, and were mounted using Vectamount (Vector).

Flow cytometry. Peripheral whole blood was taken from the retroorbital sinus using heparinized capillaries under ether anesthesia or from a tail vein using EDTA (1.6 mg/ml) as anticoagulant. Bone marrow cells (BMC) were prepared immediately after sacrifice from the proximal 1.5 cm of the tibia or femur. Bone marrow was harvested by short centrifugation of the bone. The cells were dispersed in PBS by repeated pipetting, and a single cell suspension achieved by expelling the cells through a 23 gauge syringe needle. The viability of the cells was determined by trypan blue exclusion. The percentage of viable cells was greater 95% in all samples. For labeling of cell surface hPLAP on peripheral blood and BMC, cell suspensions (approximately 10⁶ cells each) were incubated with a monoclonal mouse anti-hPLAP antibody (supernatant of clone 8B6, Dako) diluted 1:20 for 30 min on ice. Non-immune mouse IgG₁ (MOPC-21, Sigma) and cell suspensions from wild-type rats were used as negative controls. After washing twice, the cells were incubated with rat-adsorbed, FITC-labeled goat anti-mouse IgG antibody (Sigma) for 30 min on ice. Thereafter, BMC were washed with PBS and resuspended in PBS with 1% PEA prior to flow cytometric analysis. In blood samples, erythrocytes were hemolyzed after antibody staining by addition of FACS (Fluorescence-Activated Cell Sorter) lysing solution (Becton Dickinson, Heidelberg, Germany). Stained leukocytes were centrifuged, washed twice with PBS, fixed in PBS with 1% PFA, and analyzed on the flow cytometer. The analyses were performed on a FACScan flow cytometer using CellQuest Pro software (Becton Dickinson, Heidelberg, Germany) Ten-thousand events were counted in each sample.

To examine the presence of circulating anti-hPLAP antibodies in serum of wild-type rats that had previously received cells from R26-hPLAP-tg rats, wild-type and transgenic peripheral blood cells were incubated for 30 min on ice with 10 μl undiluted serum from naïve wild-type rats or from wild-type rats that had received transgenic peripheral blood leukocytes (PBL) earlier. Using FITC-labeled goat anti-rat IgG antibody (Serotec, Harwell, U K) as secondary antibody, the samples were processed and analyzed as described above.

Cell culture. To examine the expression of hPLAP in cultured osteoblastic cells, bone marrow cells from wild-type and transgenic rats were harvested from the femora and tibias, and were cultivated for osteoblastic differentiation by addition of ascorbic acid, β-glycerophosphate, and dexamethasone. After 1, 2, 3, 7, and 14 days of culture, cells were fixed with 40% ethanol for 10 minutes, washed with PBS, air-dried, and heated at 65° C. for 30 minutes. Cells were then incubated in TRIS buffer containing 0.17 mg/ml BCIP, washed, counterstained with nuclear fast red, and air-dried.

To examine expression of hPLAP in osteoclast precursor cells and mature osteoclasts, bone marrow cells were cultured in DMEM with 15% fetal calf serum in the presence of 10 nM 1,25-dihydroxy-vitamin D₃. After 7-10 days of culture, cells were fixed with 40% ethanol for 10 minutes, washed with PBS, air-dried, heated at 65° C. for 30 minutes, and stained for hPLAP activity as described above. After heating, some dishes were double-stained for tartrate resistant acid phosphatase (TRAP) activity as described (22), and, thereafter, for hPLAP enzyme activity.

Transplantation of transgenic blood and bone marrow cells into wild-type recipients. In order to determine the fate of genetically labeled donor cells in wild-type recipients, we intravenously injected 36 wild-type F344 rats with PBL or BMC (n=18 each) isolated from R26-hPLAP-tg F344 rats. Transgenic rats were killed by exsanguination from the abdominal aorta under ketamine/xylazine anesthesia. PBL were harvested using a density gradient centrifugation kit (NycoPrep 1.077A, Axis-Shield, Oslo, Norway), washed several times and resuspended in PBS. The percentage of viable cells after the isolation procedure was >90% (trypan blue exclusion). At the same time, BMC were harvested and washed as described above. After isolation, all cells were resuspended in PBS, and were kept on ice until use. Isolated genetically labeled PBL or BMC were injected intravenously at a dose of 25×10⁶ or 5×10⁶ cells per animal, respectively, into sex-matched wild-type recipients via a lateral tail vein. All recipients received daily injections of genetically labeled cells over 7 consecutive days (days 1-7). The recipient rats were killed on days 8, 15, 22, or 28 (n 4-6 each for PBL and BMC recipients) by exsanguination from the abdominal aorta under ketamine/xylazine anesthesia. From all recipient animals a variety of soft tissues and bones were harvested, fixed in 40% ethanol at 4° C. for 48 hours, and processed as described above.

Humoral and cellular immune response to transgenic cells. To examine the development of antibody production against transgenic cells, wild-type rats were repeatedly exposed to PBL from hPLAP-tg donors. The PBL were harvested from F344-Tg (R26-hPLAP) rats as described above, and resuspended in PBS. Subsequently, 1×10⁶ PBL were subcutaneously or intraperitoneally injected three times into wild-type F344 rats (1 rat each) with an 11-day interval between the injections. At baseline, at and 10 days after the last injection, blood was drawn from a tail vein of the wild-type recipients to obtain serum. For mixed lymphocyte reaction, spleen cells were isolated by density gradient centrifugation (NycoPrep 1.077A). The spleen cells from naïve wild-type rats or from wild-type rats previously exposed to transgenic cells (3 intraperitoneal injections at 10-day intervals) were co-cultured with transgenic spleen cells for 5 days at a total density of 5×10⁵ cells per well (2.5×10⁵ wild-type and trans-genic cells each). During the last 18 hours of culture, ³H thymidine (2 Ci/mmol) was added to quantify cell proliferation. ConA (10 μg/ml) was used as a positive control.

Lethal irradiation and bone marrow transplantation. To trans-plant bone marrow from F344-Tg (R26-hPLAP) rats into wild-type F344 rats, wild-type animals (n=26) were lethally irradiated with a dosage of 8.5 Gy at 0.9473 Gy/min using a cobalt-60 irradiator (Eldorado). Four hours after the irradiation, the rats were intravenously injected with 3-4 million BMC isolated from R26-hPLAP-tg rats as described above. To rule out unsuccessful engraftments, the same injection of freshly prepared transgenic BMC was repeated 24 hours after the irradiation.

Intraarticular injection of genetically labeled cells. Next, it was explored whether intraarticularly injected BMC from R26-hPLAP-tg rats would survive, proliferate, and differentiate in the knee joint of bone marrow-transplanted (BMT) wild-type rats. For this experiment 6 3-month-old male and female wild-type F344 rats were lethally irradiated and transplanted with BMC from R-26-hPLAP-tg F344 rats. Two weeks after the bone marrow transplantation, 5×10⁶ BMC isolated from F344-Tg (R26-hPLAP) rats were injected once into the cavity of the left or right knee joint. For this procedure, the recipients were anesthetized using medetomidine/midazolam/fentanyl (0.15/2.0/0.005 mg/kg i.m.). After the intraarticular injection, anesthesia was antagonized by subcutaneous injection of a mixture of atipamezole/flumazenil/naloxone (0.37/0.1/0.06 mg/kg). The rats were sacrificed 7 days or 28 days (n=3 each) after the intraarticular injection by exsanguination from the abdominal aorta under ketamine/xylazine anesthesia. The intact knee joints were harvested, trimmed, fixed in 40% ethanol at 4° C. for 48 hours, and embedded in MMA. Frontal 5-μm-thick sections were cut using a Microm HM360 microtome as described above.

Induction of tolerance by neonatal exposure to transgenic cells. To induce tolerance to the marker gene by an alternative method, 6 neonatal wild-type F344 rats pups, within 4 hours after birth, were subcutaneously injected once with 20 μl of whole blood from F344-Tg (R26-hPLAP) donors. One control litter mate did not receive transgenic cells. Four months later, all 7 rats received skin grafts (approx. 1 cm²) from sex-matched F344-Tg (R26-hPLAP) donors at the back under short anesthesia with medetomidine/midazolam/fentanyl (0.15/2.0/0.005 mg/kg i.m.). The grafts were controlled daily. Four weeks post-transplantation, biopsies were taken under anesthesia from the margin of the skin grafts, fixed in 40% ethanol at 4° C. for 48 hours, embedded in paraffin, sectioned at 5 μm thickness, and stained for hPLAP enzyme activity as described above.

Results

hPLAP enzyme activity is retained during paraffin and MMA embedding, and is better preserved using 40% ethanol as fixative FIG. 1 shows that hPLAP enzyme activity in paraffin sections of liver from R26-hPLAP-tg rats was retained during paraffin embedding, and that the enzyme activity was much better preserved using 40% ethanol (FIG. 1A) as fixative compared with 4% PFA (FIG. 1B). Endogenous alkaline phosphatase activity was totally inactivated by heating the sections at 65° C. for 30 min prior to staining (FIG. 1C). Therefore, tissue specimens were fixed with 40% ethanol in all subsequent experiments. MMA embedding did not interfere with preservation of hPLAP enzyme activity (FIG. 1E). Rather, MMA sections displayed improved morphological detail, and allowed a more precise subcellular localization of the transgene expression. For example, FIG. 1E clearly shows that the hPLAP enzyme was mainly located at the luminal membrane of renal tubular epithelium, while the staining pattern in paraffin sections was more diffuse (FIG. 1D). Similar to paraffin sections, endogenous alkaline phosphatase activity was completely inactivated in MMA sections by heat pretreatment (FIG. 1F). In addition to histochemistry, it was also possible to detect hPLAP by immunohistochemistry in PFA-fixed and ethanol-fixed paraffin- and MMA-embedded transgenic tissues, using the monoclonal anti-body 8B6 (data not shown). However, immunohistochemistry was not superior to histochemistry in terms of detection sensitivity of the marker gene. Therefore, we decided to use histochemistry for all further studies.

Ubiquitous Expression of hPLAP in Bone and Bone Marrow Cells of R26-hPLAP-tg Rats

When hPLAP expression in sections of MMA-embedded bones from R26-hPLAP-tg rats was examined, strong staining was found in all mesenchymal bone cells, i.e., in osteoblasts and osteocytes (FIG. 2A) as well as in growth plate and articular cartilage chondrocytes (FIG. 2B-C). In bone marrow, all cells expressed the transgene in R26-hPLAP-tg rats, with particularly strong cytoplasmic and membrane expression in megakaryocytes (FIG. 2D). No staining was found in bone marrow, bone or cartilage cells of wild-type rats (FIG. 2E-G). Adherent mesenchymal cells and also osteoclast precursors in bone marrow cultures of R26-hPLAP-tg rats displayed strong hPLAP expression at all stages of differentiation.

Immune Response of Wild-Type Rats After Syngeneic Transplantation of Cells from R26-hPLAP-tg Rats

In order to test whether peripheral blood cells and bone marrow cells from transgenic donors would survive and proliferate in normal wild-type rats of the same inbred strain, 25×10⁶ PBL or 5×10⁶ BMC from R26-hPLAP-tg F344 donor rats were injected daily for 1 week into wild-type F344 rats. However, histological analysis of all tissues examined (lung, liver, spleen and bone) failed to show any evidence of genetically labeled, hPLAP-expressing cells from transgenic donors in the wild-type recipients, suggesting that it was not possible to syngeneically transplant cells from transgenic donors into wild-type rats. FACS analysis revealed that almost all peripheral blood leukocytes and bone marrow cells showed membrane expression of the hPLAP enzyme in R26-hPLAP-tg rats (FIG. 3A-B). Immunization phenomena against hPLAP could result in immune-mediated destruction of any hPLAP-expressing cells in wild-type recipients, because the hPLAP enzyme represents a foreign protein to the rat immune system.

To test whether wild-type recipients develop antibodies against hPLAP after injection of cells from transgenic donor rats of the same strain, wild-type F344 rats with PBL from hPLAP-tg F344 rats were intraperitoneally or subcutaneously injected three times with an 11-day interval between the injections. Subsequently, serum from naïve wild-type F344 rats or from wild-type F344 rats that had received injections of PBL from hPLAP-tg F344 rats was incubated with PBL from transgenic rats, and the presence of antibody-labeled cells was examined by FACS analysis. As expected, serum from naïve wild-type rats that had never been in contact with transgenic cells did not react with PBL from transgenic rats (FIG. 3C). However, as shown in FIG. 3C, repeated intraperitoneal injection of transgenic PBL induced an antibody response in wild-type rats. Identical results were obtained with subcutaneous injection (data not shown). To test cellular immune responses to hPLAP in wild-type rats, mixed lymphocyte reaction protocols were performed. When spleen cells from naïve wild-type rats were incubated with transgenic spleen cells, increased cell proliferation was not observed (FIG. 3D). However, increased cell proliferation was induced when spleen cells from wild-type rats that had previously been exposed to cells from F344-Tg (R26-hPLAP) rats were incubated with transgenic cells. These data showed that exposure to hPLAP-tg cells induces immunization phenomena in wild-type rats, explaining the abovementioned failure of the experiments aimed at the syngeneic transplantation of transgenic cells into normal wild-type recipients. To verify this hypothesis, skin of F344-Tg (R26-hPLAP) rats was transplanted into naïve, 3-month-old wild-type F344 recipients (n=3). All recipients rejected the transgenic trans-plants within 2 weeks.

Induction of tolerance by bone marrow transplantation

So far, the results of the present invention showed that R26-hPLAP-tg rats are a very attractive model for regenerative therapies because of ubiquitous and strong expression of the transgene, and excellent preservation of the marker enzyme in paraffin and MMA sections. However, immune-mediated rejection of hPLAP-tg cells by wild-type rats exposed to cells from transgenic donors made it impossible to use this model to unambiguously address any of these questions. Previous studies have shown that tolerance against intracellular xenogeneic marker genes (for example GFP or neo, neomycin phosphotransferase) can be induced by busulfan treatment of wild-type C57BL/6 mice, and subsequent transplantation of bone marrow from GFP transgenic C57BL/6 mice, or by transplantation of autologous hematopoietic stem cells transduced with a neo marker gene in monkeys after total body irradiation. Based on these results, established methods of tolerance induction to a specific marker gene were combined according to the present invention with subsequent transplantation of otherwise syngeneic cells labeled with the same marker in order to avoid immune-mediated rejection of the labeled cells.

To test the idea of employing marker tolerant animals as models for regenerative therapies, lethal irradiation and transplantation of bone marrow from R26-hPLAP-tg F344 rats into wild-type F344 rats was used as proof-of-principle to prevent immune responses after injection of transgenic cells into wild-type rats. Because the rats are of the same inbred strain, graft-versus-host reactions can be ruled out a priori. FIG. 4A-D shows that in R26-hPLAP bone marrow-transplanted wild-type (R26-hPLAP BMT) rats all hematopoietic cells are of donor origin after BMT, and stably express hPLAP, 4 weeks post-transplantation. On the other hand, mesenchymal cells such as osteoblasts, osteocytes, and chondrocytes are of wild-type origin, and do not express hPLAP (FIG. 4D-E). In extraosseous tissues, this model allows to clearly differentiate between wild-type cells of recipient origin and hPLAP-expressing cells of donor origin (FIG. 4F-H). A significant portion of capillary endothelial cells was found, but not epithelial or mesenchymal cells such as muscle cells, were of donor origin in heart, kidney, and lung of R26-hPLAP BMT wild-type rats (FIG. 4F-H).

Utility of Marker Tolerant Rats for Cartilage Regeneration Studies

An example of the usefulness of marker tolerant animals for cell therapeutic studies is shown in FIG. 5. In this experiment, bone marrow cells from R26-hPLAP-tg rats were injected into the knee joint of R26-hPLAP BMT wild-type rats. One week post-injection, the transgenic BMC had formed a dense, 2-3 cells wide, layer on the intact articular cartilage surface. This finding shows that these cells underwent proliferation on the cartilage surface earlier. After 4 weeks, the genetically labeled cells displayed a more differentiated fibroblast- or chondrocyte-like phenotype and appeared to have become integrated into the surrounding cartilage tissue of wild-type origin. This experiment underscores the potential usefulness and strengths of this technology, and clearly demonstrates that mesenchymal precursors obviously present in native bone marrow can adhere to the articular cartilage surface and can, subsequently, differentiate into a fibroblastic or chondrocytic phenotype.

Neonatal Induction of Tolerance

The background of labeled hematopoietic cells is an important shortcoming of R26-hPLAP BMT wild-type rats. To test whether neonatal exposure of wild-type F344 rats with cells from F344-Tg (R26-hPLAP) rats results in a sustainable tolerance to trans-genic tissue, neonatal F344 wild-type rats (n=6) were subcutaneously injected with whole blood from F344-Tg (R26-hPLAP) rats. Four months later, skin of F344-Tg (R26-hPLAP) rats was transplanted into these neonatally tolerized rats. The transgenic skin grafts were accepted in 6 out of 6 rats, while a control littermate not having received transgenic blood promptly rejected the transgenic graft. FIG. 6A shows the margin of the trans-genic skin transplant in a marker tolerant rat, 4 weeks post-transplantation. It is evident that the border between wild-type and transgenic tissues is sharply delineated, and that capillaries from transgenic donor tissue invade the surrounding wild-type skin (FIG. 6B). This experiment demonstrates that sustainable marker tolerance can be also achieved by neonatal exposure of wild-type F344 rats to hPLAP-expressing cells of the same rat strain. Thus, any kind of hPLAP-labeled cell or tissue from F344 rats can be transplanted and studied in these marker tolerant F344 rats in the absence of immune-mediated rejection, underscoring the broad applicability of this technology for regenerative treatment protocols, cell lineage studies, and experiments requiring long-term cell tracking.

Tolerance can as outlined above, also be induced by other methods. For example, sustainable marker tolerance can be achieved by neonatal exposure of wild-type F344 rats to hPLAP-expressing cells of the same rat strain (as shown by the present example). Tolerance can also be induced by generation of hPLAP transgenic animals expressing the marker gene under the control of an inducible promoter that can be shut down after tolerance is achieved. In addition, bone marrow from animals expressing the hPLAP transgene under tissue-specific promoters targeting transgene expression to subsets of hematopoietic cells, for example to megakaryocytes, can be transplanted into lethally irradiated wild-type rats. All these approaches greatly increase the versatility of this technology by reducing the background of labeled cells in the marker tolerant animals. Because tolerance induction is also of critical importance for allogeneic and xenogeneic organ transplantations, new methods for tolerance induction will be developed in the near future. Therefore, the present invention has a very wide applicability, and will help to answer many questions related to regenerative therapies and lineage of various cell types.

### REFERENCES

1. Prockop et al., PNAS USA 2003; 100 Suppl 1:11917-23.
2. Gossler et al., In: Joyner (ed.) Gene Targeting: A Practical Approach. New York: NY Oxford Univ. Press, 1993:181-227.
3. Cubitt et al., Trends Biochem Sci 1995; 20:448-55.
4. Walter et al., Histochem J 2000; 32:99-103.
5. Zagzag et al., Brain Pathol 2003; 13:34-7.
6. Kisseberth et al., Dev Biol 1999; 214:128-38.
7. Sarukhan et al., J Gene Med 2001; 3:260-70.
8. Gross et al., Blood 2003; 102:4326-8.
9. Andersson et al., Blood 2003; 101:4305-12.
10. Heim et al., Mol Ther 2000; 1:533-44.
11. Rosenzweig et al., Blood 2001; 97:1951-9.
12. Mujtaba et al., Exp Neurol 2002; 174:48-57.
13. Spees et al., Mol Ther 2004; 9:747-56.
14. Field et al., Immunol Rev 2001; 182:99-112.
15. Ridge et al., Science 1996; 271:1723-6.
16. Shi et al., J Thromb Haemost 2004; 2:1989-97.
17. de Boer et al., Eur J Immunol 2003; 33:314-25.
18. Sasmono et al., Blood 2003; 101:1155-63.
19. Albanese et al., Semin Cell Dev Biol 2002; 13:129-41.
20. Bittira et al., Eur J Cardiothorac Surg 2003; 24:393-8.
21. Mahmood et al., Neurosurgery 2003; 53:697-702.
22. Erben et al., J. Histochem. Cytochem. 45 (1997), 307-313

1-16. (canceled)

17. A method for studying the efficacy or movement of transplanted cells in a cell therapy comprising:
obtaining cells labeled with a marker;
transplanting the labeled cells into a marker-tolerant animal; and
studying the efficacy and/or movement of the transplanted cells in a cell therapy in the marker-tolerant animal.

18. The method of claim 17, wherein the transplanted cells are allogenic to cells of the marker-tolerant animal.

19. The method of claim 17, wherein the transplanted cells are syngenic to cells of the marker-tolerant animal.

20. The method of claim 17, wherein the transplanted cells are xenogenic to cells of the marker-tolerant animal.

21. The method of claim 17, wherein the transplanted cells are non-hematopoietic cells.

22. The method of claim 21, wherein the non-hematopoietic cells are epithelial cells, endothelial cells, liver cells, pancreas cells, neuronal cells, or combinations thereof.

23. The method of claim 17, wherein the transplanted cells are mesenchymal cells.

24. The method of claim 23, wherein the mesenchymal cells are osteoblasts, chondrocytes, adipocytes, fibroblasts, myoblasts, or combinations thereof.

25. The method of claim 17, wherein the marker is a fluorogenic marker or colorigenic marker.

26. The method of claim 25, wherein the fluorogenic marker is an Escherichia coli lacZ or a green fluorescent protein (GFP).

27. The method of claim 25, wherein the colorigenic marker is a human placental alkaline phosphatase (hPLAP).

28. The method of claim 17, wherein the marker is controlled by an inducible promoter.

29. The method of claim 17, wherein the marker is controlled by a constitutive promoter.

30. The method of claim 29, wherein the constitutive promoter is R26, A-actin or 6-actin with a cytomegalovirus enhancer.

31. The method of claim 17, wherein the labeled cells are transplanted into a heart, central nervous system (CNS), bone, cartilage, joint, lung, intestine, liver, or pancreas of the marker-tolerant animal.

32. The method of claim 17, wherein the marker-tolerant animal is an inbred animal, a cloned animal, an animal transplanted with bone marrow cells expressing the marker, an animal that was exposed neonatally to the marker, an animal that was exposed postnatally to the marker by intravenous infusion, or an animal that was exposed to the marker protein by intranasal application.

33. The method of claim 17, wherein the marker-tolerant animal is a rodent, a primate, or a pig.

34. The method of claim 33, wherein the rodent is a rabbit, a hamster, a mouse, a guinea pig or a rat.

35. The method of claim 17, wherein the cell therapy is a cell therapy for stroke, multiple sclerosis, neurodegenerative disorders, arthroses, intervertebral disc degeneration, ligament injuries, myopathies, myocardial infarction, cardiomyopathies, glomerulonephritis, severe liver cell damage, epidermal cell therapy, or cancer.

36. A method of preparing a marker-tolerant animal comprising:
obtaining a transgenic animal expressing a marker protein under the control of a promoter;
isolating bone marrow cells (BMCs) from the transgenic animal;
irradiating a wild-type animal of the same species as the transgenic animal; and
injecting the isolated BMCs into the irradiated wild-type mammal, wherein a marker-tolerant animal is prepared.

37. The method of claim 36, wherein the marker-tolerant animal is a rodent, a primate, or a pig.

38. The method of claim 36, wherein the transgenic animal and the wild-type animal are from the same inbred line.

39. The method of claim 36, wherein the promoter is an inducible promoter.

40. A kit for investigating cell therapy comprising:
a marker-tolerant animal, and
a cell comprising a marker.

41. The kit of claim 40, wherein the marker-tolerant animal is a rodent, a primate, or a pig.
